SEQUENCE LISTING (3 sequences)

SEQ ID NO: 1 - protein, 12 aa, synthetic construct
GGGPQGIWGQGK

SEQ ID NO: 2 - protein, 12 aa, synthetic construct
GGGIQQWGPGGK

SEQ ID NO: 3 - protein, 12 aa, synthetic construct
GGGGGIPQQWGK

(19) United States
(12) Patent Application Publication
(10) Pub. No.: US 2026/0049177 A1
(43) Pub. Date: Feb. 19, 2026

(54) PREPOLYMERS AND COMBINATIONS THEREWITH FOR WOUND REGENERATION

(71) Applicant: SUNNYBROOK RESEARCH INSTITUTE, Toronto (CA)

(72) Inventors: Ayesha Aijaz, Toronto (CA); Marc G. Jeschke, Toronto (CA)

(73) Assignee: SUNNYBROOK RESEARCH INSTITUTE, (US)

(21) Appl. No.: 19/128,394

(22) PCT Filed: Oct. 25, 2023
(86) PCT No.: PCT/CA2023/051421

§ 371 Date: May 8, 2025

Related U.S. Application Data
(60) Provisional application No. 63/423,695, filed on Nov. 8, 2022.

(51) Int. Cl. C08G 65/48; A61K 31/728; A61K 38/01; A61K 38/39; A61K 47/60; A61P 17/02; C08J 3/075; C08J 3/24

(52) U.S. Cl. CPC C08G 65/48; A61K 31/728; A61K 38/014; A61K 38/39; A61K 47/60; A61P 17/02; C08J 3/075; C08J 3/246

(57) ABSTRACT

A polyethylene glycol (PEG) prepolymer is disclosed herein. The PEG prepolymer comprises a reaction product of an amine-reactive PEG and a protease degradable peptide. The reaction product comprises the formula [Acryl-PEG-peptide-PEGAcryl]ₙ, wherein n is greater than 0. The reaction product can be combined with a thiolated biomolecule. Matrices formed from the PEG prepolymer and the thiolated biomolecule are provided and uses thereof.

### CROSS REFERENCE TO RELATED APPLICATIONS

This application is the U.S. National Stage of International Application No. PCT/CA2023/051421, filed Oct. 25, 2023, which was published in English under PCT Article 21 (2), which in turn claims the benefit of U.S. Provisional Application No. 63/423,695, filed Nov. 8, 2022, all of which are incorporated by reference in their entirety.

### FIELD

The present invention relates to prepolymers. In particular, the present invention relates to combinations comprising prepolymers and biomolecules for wound regeneration.

### INCORPORATION OF ELECTRONIC SEQUENCE LISTING

The Sequence Listing is submitted as an XML file in the form of the file named “Sequence.xml” (3,671 bytes) created on May 5, 2025, which is incorporated by reference herein.

### BACKGROUND

Chronic wounds, such as diabetic ulcers, fistulas, and pressure sores, are a global healthcare problem. Traditional hydrogel slabs are limited in their ability to conform to irregularly-shaped wounds, which can hinder healing processes. To address this issue, injectable dressings from microparticles or crosslinkable systems have been developed; however, these systems often either fail to match the mechanical properties of native tissue or lack an interconnected porous structure, both of which can facilitate effective healing.

Moreover, dressing changes cause pain and increased infection risks, making a degradable option that can be left in place throughout healing desirable. Current research on degradable dressings is limited by the need for biocompatible degradation byproducts that do not hinder the healing process.

A need exists for the development of a product and/or method that provides the public with a useful alternative.

The background herein is included solely to explain the context of the disclosure. This is not to be taken as an admission that any of the material referred to was published, known, or part of the common general knowledge as of the priority date.

### SUMMARY OF THE INVENTION

In accordance with an aspect, there is provided a polyethylene glycol (PEG) prepolymer comprising a reaction product of an amine-reactive PEG and a protease degradable peptide, wherein the reaction product comprises the formula [Acryl-PEG-peptide-PEG-Acryl]ₙ, wherein n is greater than 0.

In aspects, the amine reactive PEG is selected from acrylate-poly (ethylene glycol)-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succinimidyl succinate (PEG-SS) or acrylate-PEG-maleimide (PEG-MAL).

In aspects, the amine-reactive PEG is acrylate-PEG-SVA.

In aspects, the peptide comprises or consists of the sequence GGGPQGIWGQGK (SEQ ID NO: 1), GGGIQQWGPGGK (SEQ ID NO: 2), or GGGGGIPQQWGK (SEQ ID NO: 3).

In aspects, the peptide comprises or consists of the sequence GGGPQGIWGQGK (SEQ ID NO: 1) or a fragment or variant thereof.

In another aspect, there is provided a combination comprising the PEG prepolymer described herein and a thiolated biomolecule.

In aspects, the thiolated biomolecule has wound regeneration and/or anti-scarring properties.

In aspects, the thiolated biomolecule comprises thiolated hyaluronic acid, thiolated gelatin, tropoelastin, or combinations thereof.

In aspects, the thiolated biomolecule can crosslink acrylate side chains of the PEG prepolymer to form a polymeric matrix.

In aspects, the PEG prepolymer and the thiolated biomolecule are to be combined prior to use, for in situ, substantially instantaneous polymerization on a surface, such as a wound bed.

In aspects, the polymerization time is about 1 second to about 30 minutes.

In aspects, the polymerization time is about 10 seconds to about 20 seconds.

In aspects, the polymerization does not require a light source and/or photoinitiator.

In aspects, the combination further comprises a photoinitiator, wherein the photoinitiator is added to the PEG prepolymer prior to combination with the thiolated biomolecule.

In aspects, the photoinitiator is selected from triethanolamine, eosin-Y, ruthenium, and n-vinylpyrrolidone and their derivatives and mixtures thereof.

In aspects, the PEG prepolymer and the thiolated biomolecule are provided separately.

In aspects, the PEG prepolymer and thiolated biomolecule are provided as separate lyophilized products for reconstitution prior to combination.

In yet another aspect, there is provided a polymeric wound matrix comprising: a polyethylene glycol (PEG) prepolymer comprising a reaction product of an amine-reactive PEG and a protease degradable peptide, wherein the reaction product comprises the formula [Acryl-PEG-peptide-PEG-Acryl]ₙ, wherein n is greater than 0; and a thiolated biomolecule; wherein the thiolated biomolecule crosslinks acrylate side chains of the PEG prepolymer to form the wound matrix.

In aspects, the amine reactive PEG is selected from acrylate-poly (ethylene glycol)-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succinimidyl succinate (PEG-SS) or acrylate-PEG-maleimide (PEG-MAL).

In aspects, the amine-reactive PEG is acrylate-PEG-SVA.

In aspects, the peptide comprises or consists of the sequence

(SEQ ID NO: 1)
GGGPQGIWGQGK,
(SEQ ID NO: 2)
GGGIQQWGPGGK,
or
(SEQ ID NO: 3)
GGGGGIPQQWGK.

In aspects, the peptide comprises or consists of the sequence GGGPQGIWGQGK (SEQ ID NO: 1) or a fragment or variant thereof.

In aspects, the thiolated biomolecule has wound regeneration and/or anti-scarring properties.

In aspects, the thiolated biomolecule comprises thiolated hyaluronic acid, thiolated gelatin, tropoelastin, or combinations thereof.

In aspects, the PEG prepolymer and the thiolated biomolecule are to be combined prior to use, for in situ, substantially instantaneous polymerization on a surface, such as a wound bed.

In aspects, the polymerization time is about 1 second to about 30 minutes.

In aspects, the polymerization time is about 10 seconds to about 20 seconds.

In aspects, the polymerization does not require a light source and/or photoinitiator.

In aspects, the matrix further comprises a photoinitiator, wherein the photoinitiator is added to the PEG prepolymer prior to combination with the thiolated biomolecule.

In aspects, photoinitiator is selected from triethanolamine, eosin-Y, ruthenium, and n-vinylpyrrolidone and their derivatives and mixtures thereof.

In aspects, the PEG prepolymer and the thiolated biomolecule are provided separately.

In aspects, the PEG prepolymer and thiolated biomolecule are provided as separate lyophilized products for reconstitution prior to combination.

In aspects, the matrix does not contract over time.

In aspects, the matrix is biodegradable.

In aspects, the matrix supports cell viability.

In aspects, the matrix further comprises cells, such as epidermal cells, including keratinocytes and fibroblasts, skin immune cells, endothelial cells, stem cells, such as induced pluripotent stem cells and mesenchymal stem cells, such as burn-derived mesenchymal stem cells and/or products derived from said cells such as a secretome or exosomes from mesenchymal stem cells.

In aspects, the matrix promotes wound regeneration and/or reduces scarring.

In aspects, the matrix promotes increased anti-scarring mediator levels, such as TGF-β3, when the matrix is placed on a wound.

In aspects, the matrix promotes granulation tissue formation and/or re-epithelization of a wound.

In aspects, the matrix promotes neovascularization of a wound.

In aspects, the matrix reduces wound inflammation.

In aspects, the matrix described herein is for wound regeneration.

In aspects, the matrix described herein is for treating and/or sealing a wound.

In aspects, the wound comprises burns, partial and full-thickness wounds, acute wounds, chronic wounds, pressure ulcers, venous ulcers, arterial ulcers, diabetic ulcers, chronic vascular ulcers, draining wounds, tunneled or undermined wounds, surgical wounds, donor site wounds, or, trauma wounds.

In aspects, the matrix described herein is in the form of a biodegradable wound dressing or a biodegradable or non-biodegradable donor site dressing.

In aspects, the matrix is a hydrogel.

In yet another aspect, there is provided a method of treating and/or sealing a wound comprising applying the combination described herein or polymeric matrix described herein to the wound.

In aspects, the wound comprises burns, partial and full-thickness wounds, acute wounds, chronic wounds, pressure ulcers, venous ulcers, arterial ulcers, diabetic ulcers, chronic vascular ulcers, draining wounds, tunneled or undermined wounds, surgical wounds, donor site wounds, or, trauma wounds.

In yet still another aspect, there is provided a use of the combination described herein or the polymeric wound matrix described herein for treating and/or sealing a wound.

In aspects, the wound comprises burns, partial and full-thickness wounds, acute wounds, chronic wounds, pressure ulcers, venous ulcers, arterial ulcers, diabetic ulcers, chronic vascular ulcers, draining wounds, tunneled or undermined wounds, surgical wounds, donor site wounds, or, trauma wounds.

In still yet another aspect, there is provided a bio-ink formulation comprising the combination described herein, wherein the bio-ink formulation is used for skin regeneration.

In still another aspect, there is provided a method of making the polymeric matrix described herein, comprising combining the PEG prepolymer described herein with the thiolated biomolecule described herein and crosslinking acrylate side chains of the PEG prepolymer with the thiolated biomolecule to form the polymeric matrix.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIGS. 1A-1D show a set of graphs depicting the material characterization of a PEG hydrogel PEGScarX. (FIGS. 1A-1B) Storage modulus (G′) and loss modulus (G″) of PEGScarX. (FIG. 1C) Quantification of degradation in exogenous collagenase. (FIG. 1D) Swelling kinetics in PBS.

FIGS. 2A-2G show various images depicting cell biocompatibility of the hydrogel PEGScarX. (FIGS. 2A-2B) Side and top view images of cell viability of burn-derived mesenchymal stem cells (BDMSCs) embedded in PEGScarX. Bright field micrographs of cell spreading on (FIG. 2C) day 5 and (FIG. 2D) d21 post-encapsulation. Immunofluorescence micrographs of (FIG. 2E) cell spreading using actin staining and ECM deposition (FIG. 2F) collagen and (FIG. 2G) laminin deposition.

FIG. 3 shows a pictograph of the contraction and degradation of PEGScarX over time. Collagen fibers are depicted in the inset image after 7 and 14 days.

FIG. 4 shows graphs depicting amounts of factor secreted from burn-derived mesenchymal stem cells (BDMSCs) embedded in PEGScarX hydrogels.

FIGS. 5A-D show wound histological images and re-epithelization ability of PEGScarX treated wounds. (FIG. 5A) H & E and Masson's trichrome histological sections (inset depicts image of collagen fibers). (FIG. 5B) Quantification of wound re-epithelialization kinetics. (FIG. 5C) Quantification of collagen density. (FIG. 5D) Quantification of number of rete ridges.

FIGS. 6A-6E show the results of wound scar assessment using PEGScarX. (FIG. 6A) Gross appearance of wound scar (top) and expression of a-SMA (bottom). (FIG. 6B) Vancouver Scar Scores of the wounds of A. (FIG. 6C) Quantification of a-SMA positive area in wounds. (FIG. 6D, 6E) Quantification of TGF-β3 levels in wounds.

FIGS. 7A-7D show the results of PEGScarX involvement in neovascularization. (FIG. 7A) Immunohistochemical analysis of neovascularization using anti-CD31 to stain for blood vessels. (FIG. 7B) Quantification of the number of blood vessels in each of wounds of FIG. 7A. (FIG. 7C) Western blot analysis of VEGF in wound sections. (FIG. 7D) Quantification of VEGF expression level in each of the wounds of FIG. 7A.

FIG. 8 shows the results of PEGScarX involvement in wound inflammation. IHC analysis of CD11b and quantification of CD11b+ cells in each of the wounds.

FIGS. 9A-9C show the material characterization of donor site dressings in accordance with the present disclosure. (FIG. 9A) Gross appearance of donor site dressings before and after swelling in PBS. Scale bars=1 cm. (FIG. 9B) Swelling kinetics. (FIG. 9C) Swelling ratios.

FIGS. 10A-10C show assessment of donor site wounds treated with donor site dressings in accordance with the present disclosure. (FIG. 10A) Gross appearance of donor site wounds. (FIG. 10B) Donor site wound healing kinetics. (FIG. 10C) Vancouver scar scales for donor site wound scar quality demonstrate scar less healing with PEG-tHG dressings.

FIGS. 11A-11C show histological images and collagen density of wounds treated with donor site dressings in accordance with the present disclosure. (FIG. 11A) H & E and trichrome sections. (FIG. 11B) Quantification of rete ridges. (FIG. 11C) Quantification of collagen density.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in some aspects, polymerization does not require a light source and/or photoinitiator.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “wound” as used herein indicates the result of a disruption of normal anatomic structure and function of an individual ((1988 (November), 1998 (May), 2007 (February)) (Lazarus, Cooper et al. 1994). Accordingly, wounds in the sense of the disclosure encompass a wide range of a defects or breaks in a tissue and/or organs of an individual, resulting from physical, chemical and/or thermal damage, and/or as a result of the presence of an underlying medical or physiological condition as will be understood by a skilled person (Boateng, Matthews et al. 2008)). Exemplary wounds within the scope of the present disclosure are provided in the description below.

The term “wound healing” as used herein indicates a biological process directed to growth and tissue regeneration in the individual (Boateng. Matthews et al. 2008) In particular, during the wound healing process cellular and extracellular components of the injured tissue or organ interact to restore the integrity of the organ or tissue in interdependent and overlapping stages will be understood by a skilled person (Strodtbeck 2001) (Russell 2000) (BA 1999) (GS 1999) (Rothe and Falanga 1989), and (Shakespeare 2001)). In particular, a wound heling process in the sense of the disclosure comprises hemostasis, inflammation, migration, proliferation and maturation phases (GS 1999) (DM 1999)).
i. The term “hemostasis” in the sense of the disclosure indicates a stage of wound healing characterized by the presence of by exudate (blood without cells and platelets), exudate components such as clotting factors, coagulation of the exudate, formation of a fibrin network, and production of a clot in the wound causing bleeding to stop (Boateng, Matthews et al. 2008) (Martin 1997);
ii. The term “inflammation” in the sense of the disclosure indicates a stage of wound healing process characterized by release of protein-rich exudate, vasodilation through release of histamine and serotonin, presence of phagocytes and engulf dead cells forming necrotic tissue in the wound, sloughy (yellowish colored mass), and platelets aggregate as will be understood by a skilled person (Boateng, Matthews et al. 2008)). The inflammatory phase occurs almost simultaneously with hemostasis, sometimes from within a few minutes of injury to 24 hours and lasts for about 3 days as also understood by a skilled person. (Boateng, Matthews et al. 2008));
iii. The term “migration” in the sense of the disclosure indicates a stage of wound healing process characterized by movement of epithelial cells and fibroblasts to the injured area, regeneration and growth of fibroblast and epithelial cells accompanied by epithelial thickening. (Boateng, Matthews et al. 2008));
iv. The term “proliferation” in the sense of the disclosure indicates a stage of wound healing process characterized by formation of granulation tissue, collagen synthesis and in-growth of capillaries and lymphatic vessels into the wound, formation of blood vessels, fibroblast proliferation and collagen thickening blood vessels decrease and oedema recedes, as will be understood by a skilled person. (Boateng, Matthews et al. 2008)). The proliferative phase occurs almost simultaneously or just after the migration phase (Day 3 onwards) and basal cell proliferation, which lasts for between 2 and 3 days, and continues for up to 2 weeks by which time blood vessels decrease and oedema recedes as will also be understood by a skilled person. (Boateng, Matthews et al. 2008)); and,
v. The term “maturation” or “remodeling” in the sense of the disclosure indicates a stage of wound healing process characterized by formation of cellular connective tissue and strengthening of the new epithelium which determines the nature of the final scar. (Boateng, Matthews et al. 2008)) Cellular granular tissue is typically changed to an acellular mass from several months up to about 2 years.
   In aspects, the matrices described herein are capable of impact at least one of hemostasis, inflammation, migration, proliferation and maturation phases.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Variants” are biologically active constructs, proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments, for example of at least 10 amino acids, that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The terms “therapeutically effective amount”, “effective amount” or “sufficient amount” mean a quantity sufficient, when administered to a subject, including a mammal, for example a human, to achieve a desired result, for example an amount effective to treat and/or seal a wound. Effective amounts of the polypeptides described herein may vary according to factors such as the condition in question, age, sex, and weight of the subject. Dosage or treatment regimes may be adjusted to provide the optimum therapeutic response, as is understood by a skilled person. For example, administration of a therapeutically effective amount of the combination described herein is, in aspects, sufficient to promote wound regeneration and/or to reduce scarring.

Moreover, a treatment regime of a subject with a therapeutically effective amount may consist of a single administration, or alternatively comprise a series of applications. The length of the treatment period depends on a variety of factors, such as the condition in question, the age of the subject, the concentration of the components in the combination, the responsiveness of the patient to the components in the combination, or a combination thereof. It will also be appreciated that the effective dosage of the combination used for the treatment may increase or decrease over the course of a particular treatment regime. Changes in dosage may result and become apparent by standard diagnostic assays known in the art. The combination described herein may, in aspects, be administered before, during or after treatment with conventional therapies for the disease or disorder in question, such as a wound.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

### PEG Prepolymers and Thiolated Biomolecules

Described herein are polyalkylene glycol prepolymers, and, in particular aspects, polyethylene glycol (PEG) prepolymers. The polyethylene glycol (PEG) prepolymer comprises a reaction product of an amine-reactive PEG and a protease degradable peptide. The reaction product comprises the formula [Acryl-PEG-peptide-PEG-Acryl]ₙ, wherein n is greater than 0. As would be understood, n can be any natural number (e.g. all positive integers from 1 till infinity).

The amine reactive PEGs have acrylate side chains, such that the PEG prepolymers described herein are acrylated. For greater clarity, the term “acrylate” includes both acrylate and methacrylate and the term “acrylated” includes both acrylated and methacrylated. In other words, the amine reactive PEGs have acrylate side chains, such that the PEG prepolymer is acrylated, or the amine reactive PEGs have methacrylate side chains, such that the PEG prepolymer is metacrylated. Based on the foregoing formula, it contemplated that the reaction product can comprise acrylate side chains, methacrylate side chains or a combination thereof.

The amine reactive PEG includes, but is not limited to, acrylate-poly (ethylene glycol)-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succinimidyl succinate (PEG-SS) or acrylate-PEG-maleimide (PEG-MAL). In typical aspects, the amine-reactive PEG is acrylate-PEG-SVA.

The protease degradable peptide comprises or consists of the sequence GGGX₁X₂X₃X₄X₅X₆X₇GX₈, wherein X₁ and X₄ are independently selected from a non-polar amino acid (e.g. alanine (A), valine (V), leucine (L), isoleucine (I), methionine (M), phenylalanine (F), tyrosine (Y), tryptophan (W), proline (P), glycine (G) or cysteine (C)); in typical aspects, X₁ is isoleucine (I), such that a cleavage site may be between glycine (G) and isoleucine (I) in the sequence; and wherein X₂, X₃, X₅, X₆ and X₇ are independently selected from a non-polar amino acid or polar amino acid (e.g. (aspartic acid (D), glutamic acid (E)), serine(S), threonine (T), asparagine (N), glutamine (Q)); and wherein X₈ is selected from an amino acid having a positively charged side chain (e.g. arginine (R), histidine (H) or lysine (K)). The protease degradable peptide may be selected based on the peptide's ability to link via the amine reactive groups of the PEG. This linking ability is typically tied to the presence of the amino acid having positively charged side chains. In typical aspects, this amino acid is lysine (K).

In aspects, the non-polar amino acid is proline (P), isoleucine (I), tryptophan (W) and/or glycine (G), and in other typical aspects, the polar amino acid is glutamine (Q). The sequence of the protease degradable peptide can depend on a number of factors, including controlling and/or tailoring the degradation rates of the matrix (e.g. hydrogel). Modifying the sequence and testing the degradation rate using conventional protein degradation assays would be understood and employable by the skilled person art.

In specific aspects, the protease degradable peptide comprises or consists of the sequence GGGPQGIWGQGK (SEQ ID NO: 1), GGGIQQWGPGGK (SEQ ID NO: 2), or GGGGGIPQQWGK (SEQ ID NO: 3). In further specific aspects, the protease degradable peptide comprises or consists of GGGPQGIWGQGK (SEQ ID NO:1), a fragment or a variant thereof. In aspects, a biologically active variant will have an amino acid sequence with at least about 70% amino acid sequence identity with the comparative sequence, such as at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the comparative sequence.

The PEG prepolymer described herein is combinable with biomolecules that are capable of crosslinking with the reaction product of the PEG prepolymer. Thus, in aspects, a combination comprising the PEG prepolymer described herein and a thiolated biomolecule is provided. In this way, the biomolecule, prior to combination with the PEG prepolymer is converted into a thiol, the latter of which helps the biomolecule crosslink with the acrylate side chains of the reaction product. Methods of chemically modifying the biomolecule, such as to covalently attach one or more thiol (SH) moieties to the biomolecule, are known in the art, and can be accomplished using known methods.

The biomolecules described herein, such as the thiolated biomolecule described herein, are typically conducive to wound regeneration and/or have anti-scarring properties. Thus, biomolecules that assist with improvement of wound outcomes are contemplated within the scope of the present disclosure. These include, but are not limited to, hyaluronic acid, gelatin, tropoelastin, elastin, collagen, alginate, chitosan, chondroitin sulfate, pectin, cellulose, carboxymethylcellulose, corticosteroids, B-Complex vitamins (e.g. thiamine (B1), riboflavin (B2), nicotinamide and nicotinic acid (B3), pantothenic acid (B5), pyridoxine and pyridoxal (B6), biotin (B7), folic acid (B9), cobalamins (B12)), or combinations thereof.

In typical aspects, the thiolated biomolecule comprises thiolated hyaluronic acid, thiolated gelatin, tropoelastin, or combinations thereof. When solutions of the thiolated biomolecules described herein are prepared for combination with the PEG prepolymer described herein, the thiolated hyaluronic acid or the thiolated gelatin can have a concentration of about 0.5 mg/mL to about 3 mg/mL, such as, from about 0.5 mg/mL, about 1 mg/mL, about 1.5 mg/mL, about 2 mg/mL or about 2.5 mg/mL, to about 1 mg/mL, about 1.5 mg/mL, about 2 mg/mL, about 2.5 mg/mL or about 3 mg/mL. In typical aspects, the concentration is about 1 mg/mL. In a similar aspect, when a solution of the thiolated biomolecule is prepared, the tropoelastin therein can have a concentration of about 5 μg/mL to about 45 μg/mL, such as, from about 5 μg/mL, about 10 μg/mL, about 15 μg/mL, about 20 μg/mL, about 25 μg/mL, about 30 μg/mL, about 35 μg/mL, or about 40 μg/mL, to about 10 μg/mL, about 15 μg/mL, about 20 μg/mL, about 25 μg/mL, about 30 μg/mL, about 35 μg/mL, about 40 μg/mL, or about 45 μg/mL. In typical aspects, the concentration of the tropoelastin is about 25 μg/mL. In typical aspects, a separate solution of the PEG prepolymer described herein and the thiolated biomolecule described herein are combined and are polymerized as described below.

The thiolated biomolecule described herein can act as a crosslinking component in the combination described herein. In typical aspects, the combination described herein results in the formation of a polymeric matrix, which, based on the wound regeneration and/or have anti-scarring properties of the biomolecule, the polymeric matrix can be used in treating a wound (described in further detail below). In aspects, the thiolated biomolecule can crosslink the acrylate side chains of the PEG prepolymer to form the polymeric matrix. In specific aspects, the thiolated biomolecule and the PEG prepolymer are to be combined prior to use, for in situ, substantially instantaneous polymerization on a surface, such as a wound bed. In situ, substantially instantaneous polymerization on the wound, for example, may allow the polymeric matrix described herein to accommodate complex wound contours since the polymerization is taking place directly on the wound.

The polymerization takes place in a certain amount of time. The amount of time is form about 1 second to about 30 minutes, such as from about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 25 seconds, about 30 seconds, about 35 seconds, about 40 seconds, about 45 seconds, about 50 seconds, about 55 seconds, about 60 seconds, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, or about 29 minutes, to about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 25 seconds, about 30 seconds, about 35 seconds, about 40 seconds, about 45 seconds, about 50 seconds, about 55 seconds, about 60 seconds, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes or about 30 minutes.

In typical aspects, the polymerization time is about 1 second to about 1 minute, and in further typical aspects, the polymerization time is about 10 seconds to about 20 seconds. The speed of polymerization may be altered by, for example, changing the solvent used to dissolve the prepolymer described herein. The ability to control the rate of polymerization may be useful for applications, such as bio-printing, that can require slower polymerization times, or in the case of faster polymerization times, the increased speed of polymerization can result in the production of a matrix that is immediately accessible post-injury and easy to apply (e.g. an unskilled person can mix the two components together), thereby starting the wound regeneration/healing process within minutes.

In aspects, the polymerization does not require a light source and/or photoinitiator. Modified synthetic biomaterials such as acrylated gelatin, collagen and polyethylene glycol (PEG) typically require exposure to ultraviolet or visible light and addition of toxic photo-initiators to form hydrogel matrices. The combination described herein, however, does not require such toxic photoinitiators and/or exposure to light to result in the polymerization described herein. In this way, there is no need for a specialized setting, equipment and/or further reagents, as the PEG prepolymer and the thiolated biomolecule are in situ polymerizable once combined. The polymerization typically occurs through a thiol-Michael addition click reaction, the reaction of which is understood in the art. Thus, the polymerization of the combination described herein may allow for critical, early-stage trauma care at the point of injury or a pre-hospital setting by non-specialized service members.

In other aspects, the PEG prepolymer is photopolymerizable. Thus, in these aspects, the combination described herein further comprises a photoinitiator. The photoinitiator is added to the PEG prepolymer described herein prior to combination with the thiolated biomolecule described herein. The photoinitiator can be selected from any suitable photoinitiator to assist with the polymerization process, such as, for example, triethanolamine, eosin-Y, n-vinylpyrrolidone, ruthenium, their derivatives and mixtures thereof.

Regardless of whether or not the polymerization requires a light source and/or photoinitiator, the PEG prepolymer described herein and the thiolated biomolecule described herein can be combined in the combination described herein in any suitable ratio, such as 4:1, 3:1, 2:1, 1:1, 1:2, 1:3 or 1:4. In typical aspects, the PEG prepolymer described herein and the thiolated biomolecule described herein are combined at a ratio of about 1:1. When the polymerization requires the photoinitiator, the photoinitiator described herein is added to the PEG prepolymer prior to combination with the thiolated biomolecule described herein and the polymerization reaction comprises exposure to white light for about 5 seconds to about 40 seconds, such as, from about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 25 seconds, about 30 seconds, or about 35 seconds, to about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 25 seconds, about 30 seconds, about 35 seconds, or about 40 seconds. In typical aspects, the polymerization reaction comprises exposure to white light for about 20 seconds.

In aspects, the PEG prepolymer described herein and the thiolated biomolecule described herein are provided separately. In this way, the PEG prepolymer described herein and the thiolated biomolecule can be provided as, for example, separate lyophilized products or as water-free concentrates in containers. Suitable containers include, for example, bottles, vials, Eppendorf™ tubes and test tubes. The containers may be formed from a variety of materials such as glass or plastic. These separate lyophilized products can be reconstituted in, for example, sterile water, or saline, prior to use (e.g. prior to their combination with each other). In typical aspects, the PEG prepolymer would be reconstituted to provide an about 5% (w/v) to about 20% (w/v) solution, such as, from about 5% (w/v), about 10% (w/v), or about 15% (w/v), to about 10% (w/v), or about 15% (w/v), or about 20% (w/v) solution, which can then be combined with the reconstituted thiolated biomolecule. In typical aspects, the PEG prepolymer would be reconstituted to provide an about 10% (w/v) solution. The combination described herein provided as separate reconstitutable components may overcome limitations associated with specialized storage conditions, transport, preparation and/or requirement of a sterile surgical facility.

The PEG prepolymer described herein and the thiolated biomolecule described herein may also be combined together and polymerized as described herein into sheets, which can be subsequently packaged as, for example, off-the-shelf products, for direct application onto a wound bed. This is similar to commercial wound templates and dressings, such as those of Allevyn™ and Xeroform™ or Integra™. In this way, the combination described herein, in the form of sheets, for example, can allow for the production of matrices for a number of different sized wound areas since the formed polymeric matrices can be prepared and shaped as desired.

The combination described herein may be formulated as a bio-ink formulation. The term “bio-ink formulation” is used to mean a formulation/composition comprising the components as disclosed herein. The bio-ink formulation denotes a formulation that is used as an ink for printing a bio-printed skin-substitute using a 3D printer. A bio-printed skin substitute can be obtained by printing the bio-ink formulation on a scaffold using a 3D printer. The dimensions of the bio-printed skin may vary as per the requirements of the subject in need thereof. In aspects, the bio-printed skin may be used to cover the entire wounded area.

In aspects, the bio-ink formulation comprising the combination described herein is not cross-linked completely, and as such the bio-ink formulation may comprise the photoinitiator described above, which can start the cross-linking process in the presence of light, such as high intensity white light. The complete cross-linked bio-ink may also be referred to as a hydrogel as described herein. In these aspects, the bio-printed skin substitute may be obtained by printing the bio-ink formulation on a scaffold, followed by exposure to high-intensity white light for complete cross-linking to take place. In other aspects, exposure to high-intensity white light is not required for the cross-linking to take place. Thus, in aspects, the bio-ink formulation comprises the combination described herein and the combination is cross-linked completely, such that the photoinitiator described above is not required to be comprised in the bio-ink formulation. To this regard, the bio-ink formulation does not comprise and/or use the photopolymerizable PEG prepolymer as described above. In these aspects, the PEG prepolymer described herein can be sequentially extruded with the thiolated biomolecules described herein, one after the other. In aspects, the crosslinking time to extrude both PEG prepolymer described herein and thiolated biomolecules described herein can be tuned. Thus, the bio-ink formulation comprising the combination described herein can be useful for skin regeneration/substitution.

In a further aspect, a commercial kit is provided comprising the PEG prepolymer described herein and the thiolated biomolecule described herein. A kit comprises suitable packaging material and the combination described herein, wherein the PEG prepolymer described herein and the thiolated biomolecule described herein are provided separately as described herein for subsequent polymerization as described herein. The kit may be contained within packaging. The kit may further comprise other materials desirable from a commercial and user standpoint, including buffers, diluents, filters, needles, syringes and package inserts with instructions for use.

### Matrices

Also described herein are polymeric wound matrices. The polymeric wound matrix comprises the PEG prepolymer comprising the reaction product of the amine-reactive PEG and the protease degradable peptide, wherein the reaction product comprises the formula [Acryl-PEG-peptide-PEG-Acryl]ₙ, wherein n is greater than 0; and the thiolated biomolecule. The thiolated biomolecule crosslinks acrylate side chains of the PEG prepolymer to form the wound matrix. In other aspects, methods of making the polymeric wound matrix are also provided. The method comprises combining the PEG prepolymer described herein with the thiolated biomolecule described herein and crosslinking the acrylate side chains of the PEG prepolymer with the thiolated biomolecule to form the polymeric matrix. It is noted that any reference to the PEG prepolymer, the amine-reactive PEG, the protease degradable peptide, the reaction product, the thiolated biomolecule, the acrylate sides and the polymerization thereof are the same as described above, and therefore will not be reiterated in this section for the sake of brevity and avoidance of repetition in the description.

In aspects, the matrix described herein does not contract over time. Collagen-containing matrices typically contract over time, however, the matrices described herein typically do not contract (e.g. reduction in area). This can be a beneficial feature of the matrix described herein since the matrix can effectively maintain it's shape and/or size and thereby continue to cover and protect the wound. Moreover, lack of contraction of the matrix can also provide a greater surface area (as compared to the surface area of a contracted matrix) which can allow for, for example, cells from the wound edges to migrate and infiltrate the wound, proliferate, secrete growth factors, chemokines, cytokines, and the like, and attain their characteristic cell morphology. In this way, the cells and granulation tissue may be able to achieve their characteristic biophysical and mechanical properties as close to the native tissue, which may also contribute to the prevention of wound contraction. In this way, the matrix can continue to facilitate wound regeneration as well as protect the subject from, for example, microbial infections, by acting as a barrier thereon.

The matrix described herein is typically biodegradable. Thus, the matrices described herein, either after placement on the wound, or after in situ, substantially instantaneous polymerization on the wound, as described herein, can degrade over time, such that removal or dressing changes of the matrix are typically not required. In this way, the matrix described herein may be able to integrate into the wound bed, which can allow for the formation of new tissue, and/or reduce the requirements of further dressing changes. Thus, in aspects, the matrix described herein is dissolvable and replaceable by native tissue since the matrix can contribute to wound regeneration, such as re-epithelialization, as described herein. In this way, the matrix described herein may support skin regeneration. Moreover, secondary inventions or autografting may not be necessary at later stages of treatment with the matrix described herein since the biodegradable matrix can promote wound re-epithelialization, and prevent scar formation as described herein.

In other aspects, the matrix described herein is atramatically removable. This aspect of the matrix is typically observed when non-degradable PEG is used to form the matrix described herein such as for donor site dressings, etc. This feature of the matrix can be achieved because non-degradable PEG does not allow cell attachment or protein adsorption onto the matrix and is non adhesive to tissue with high water content.

In other aspects, the matrix described herein supports cell viability. Cells that are embedded in the matrix described herein, such as, for example, stem cells, do not typically have hypoxic centers or cell death. Moreover, the matrix described herein is typically conducive to cell adhesion and/or cell spreading. Thus the matrices described herein are non-toxic and additionally can promote, for example, the growth and proliferation of cells that are embedded therein, or in contact therewith (e.g. from the surrounding uninjured skin), which may be used to further facilitate wound regeneration, through cellular proliferation and/or the production of growth factors that assist with wound repair.

In further aspects, the matrix may further comprise cells. In typical aspects, the cells are embedded within the matrix described herein. The cells may be, for example, epidermal cells, including keratinocytes and fibroblasts, skin immune cells, endothelial cells and stem cells. The cells are typically stem cells, including, but not limited to, induced pluripotent stem cells, and mesenchymal stem cells (MSCs), such as, for example, burn-derived mesenchymal stem cells, human bone marrow-derived mesenchymal stem cells, adipose tissue-derived mesenchymal stem cells, umbilical cord-derived mesenchymal stem cells, Wharton jelly-derived mesenchymal stem cells, and dental pulp derived mesenchymal stem cells. In typical aspects, the stem cells are burn-derived mesenchymal stem cells, and typically, the burn-derived mesenchymal stem cells are useful in contributing the features of the matrices described herein, such as, wound healing, neoangiogenesis, anti-scarring and/or wound contracture. In aspects, about 1×10⁶ to about 1×10⁹ cells can be embedded within the matrix described herein. In other aspects, the matrix further comprises stem cell products such as, for example, a secretome or exosomes from the MSCs described herein.

The produced polymeric matrix described herein is typically a hydrogel, and therefore the PEG prepolymer and the thiolated biomolecule may be considered hydrogel prepolymer components. As the hydrogel comprises a network of polymer chains that are hydrophilic in nature, the hydrogel is capable of absorbing a high volume of water or other aqueous solution. In aspects, the hydrogels comprises at least 70% v/v water, at least 80% v/v water, at least 90% v/v water, at least 95%, 96%, 97%, 98% and even 99% or greater v/v water (or other aqueous solution). In aspects, the hydrogel possesses a degree of flexibility very similar to natural tissue (e.g. due to the water content). Thus, when the hydrogel is formed using the PEG prepolymer and the thiolated biomolecule described herein, the aforementioned flexibility of the hydrogel may allow the hydrogel to conform to/mold to the contours of the wound. This may be the result of the hydrogel produced in situ, as described herein, or any other hydrogel produced with the polymerization process described herein. In typical aspects, the hydrogel biodegradable. Thus, the hydrogel can dissolve on the wound and leave healed tissue in it's place.

The matrix described herein may take the form of, and/or be comprised in, for example, a biodegradable wound dressing, a biodegradable (or non-biodegradable) donor site dressing, a biodegradable gauze, a biodegradable bandage, a biodegradable pad, a biodegradable foam dressing, a biodegradable film dressing, a biodegradable sheet or a biodegradable patch. In typical aspects, the matrix described herein takes the form of and/or is comprised in a biodegradable wound dressing or a biodegradable (or non-biodegradable) donor site dressing.

The matrices described herein are absorbent. In aspects, the matrices described herein have an absorption capacity of about 10 fold to about 40 fold increase a dry weight of the matrix. For example, the absorption capacity can be about 10, about 15, about 20, about 25, about 30, about 35, or about 40, fold increase the dry weight of the matrix formed by the PEG prepolymer described herein and the thiolated biomolecule described herein. This absorptive capacity can contribute to the ability of the matrices described herein to, for example, absorb wound exudate. In aspects, the matrix described herein is capable of absorbing wound exudate while maintaining oxygen permeability.

### Methods and Uses Thereof

The matrices described herein, produced from the PEG prepolymer described herein and the thiolated biomolecule described herein are typically used for treating wounds. Typically, the wound or injury is with respect to a skin surface. The wounds include, but are not limited to, burns, partial and full-thickness wounds, acute wounds, chronic wounds, pressure ulcers, venous ulcers, arterial ulcers, diabetic ulcers, chronic vascular ulcers, draining wounds, tunneled or undermined wounds, surgical wounds, donor site wounds, or trauma wounds. In typical aspects, the surgical wounds include, but are not limited to donor sites/grafts, post-Mohs surgery, post-laser surgery, podiatric dehiscence, and wound dehiscence. In further typical aspects, the trauma wounds include, but are not limited to, abrasions, lacerations, first, second, or third degree burns, and skin tears.

The matrices described herein allow for a non-surgical, ready-to-use treatment strategy for assisting in wound repair through the promotion of tissue regeneration or augmentation while minimizing scarring and risk of infection. Thus, in aspects, the matrix described herein promotes wound regeneration and/or reduces scarring. The reduction in scarring can be seen through increased levels of anti-scarring mediators, such as, for example, TGF-β3, levels found in the wound site. In this way, when the matrix is placed on the wound, or when the matrix is formed in situ, for substantially instantaneous polymerization on the wound as described herein, scar development as noted visually and/or as measured with, for example, the Vancouver Scar Score, is reduced.

In aspects, the matrix described herein promotes granulation tissue formation and/or re-epithelization of a wound. This can be beneficial to the subject to which the matrix is applied as increased development of granulation tissue and/or migration of epithelial and/or subcutaneous tissues may result in faster healing times. The term “migration” is typically related to the stage of wound healing process characterized by (i) movement of epithelial cells and fibroblasts to the injured area, (ii) regeneration and growth of fibroblast and epithelial cells accompanied by (iii) epithelial thickening. In view of the foregoing, the matrices described herein, applied to the wound, may be able to promote at least one of (i), (ii) and (iii).

In other aspects, the matrix described herein promotes neovascularization of a wound. In this way, the matrices described herein are capable of impacting the “prefoliation” stage of wound healing through, for example, in-growth of capillaries and lymphatic vessels into the wound, and/or formation of blood vessels. In this way, the matrices described herein are capable of promoting angiogenesis at the wound site which can support to the migration of other cells, like epithelial cells to the wound site and thus the formation of new tissue thereon. In this way, the biodegradable matrix described herein can be seen to provide a scaffold for endogenous cellular invasion/infiltration and capillary growth.

In other aspects, the matrix may also carry and/or allow for the production of growth factors and cytokines, which can promote, for example, fibroblast cell migration and proliferation, to the wound site. In further aspects, the matrix may also include biofactors that support anti-scarring and/or skin regeneration. These biofactors, include, for example, hyaluronic acid, gelatin, tropoelastin, elastin, collagen, alginate, chitosan, chondroitin sulfate, pectin, cellulose, carboxymethylcellulose, corticosteroids, B-Complex vitamins (e.g. (thiamine (B1), riboflavin (B2), nicotinamide and nicotinic acid (B3), pantothenic acid (B5), pyridoxine and pyridoxal (B6), biotin (B7), folic acid (B9), cobalamins (B12)) or combinations thereof.

In aspects, the matrix described herein reduces wound inflammation. Inflammation in wound healing may be associated with the presence of phagocytes and engulf dead cells forming necrotic tissue in the wound. A reduction in the inflammatory response elicited by the wound, through the use of the matrices described herein, may result in a reduction in, for example, the amount of necrotic tissue in the wound, as fewer proinflammatory cells, like the aforementioned phagocytes make their way to the wound. Less necrotic tissue may contribute to less scar formation, which is an aforementioned benefit, in aspects, of the matrices described herein. Thus, as the matrices described herein can be seen as bioactive and/or bioinstructive wound matrices that can assist and/or promote wound healing when placed upon the wound, or after the matrices is produced in situ (e.g. substantially instantaneous polymerization on the wound), such that the matrices described herein are useful for wound regeneration.

Also described herein are methods and uses of the matrices described herein. In aspects, a method of treating a wound is provided. In other aspects, a method of sealing a wound is provided. The method comprises applying the polymeric matrix described herein to the wound. The applying can be for example, laying the matrix on the skin or wound (e.g., covering the skin or wound with the matrix), inserting the matrix into the wound (e.g., to fill the wound with the matrix), in situ polymerizing the matrix on and/or in the wound surface, or adhering the matrix to the skin or wound (e.g., the adhering may be through a medical-grade adhesive applied to the surface of the wound, followed by application of the matrix to the adhesive, or application of the medical-grade adhesive to the matrix, followed by application of the matrix to the wound). Thus, application of the matrices described herein to the wound can not only seal the wound but can also prevent further infection, by closing/sealing the wound, and/or by the matrix acting as a barrier to prevent infiltration of microorganisms into the wound site and/or bacterial biofilm formation.

In other aspects, a method of forming the polymeric matrix described herein, in situ, for treating a wound is also provided. The method comprises combining, prior to use, the PEG prepolymer described herein and the thiolated biomolecule described herein for in situ, substantially instantaneous polymerization on the wound as described herein. In other aspects, the combination of the PEG prepolymer described herein and the thiolated biomolecule described herein is not in situ, and therefore the polymerization can be used to form, for example, sheets that can be packaged for later use (e.g. for subsequent application to the wound and treatment of the same).

Also contemplated are uses of the matrices described herein. In aspects, the use of the matrix described herein is for treating a wound. In other aspects, the use of the combination described herein, in situ, for treating a wound is also provided. In this aspect, prior to use, the PEG prepolymer and the thiolated biomolecule are combinable for in situ, substantially instantaneous polymerization on the wound as described herein. In typical aspects, the matrix is for topical administration to the wound such as by either placing a pre-formed matrix on the wound, or by forming the matrix in situ, by substantially instantaneous polymerization as described herein. Since the matrix is topically applied on skin wounds, the thickness of the matrix is related to mL/cm². For example, about a 1 mL/cm² wound area to achieve a matrix thickness of about 1 mm. Thus, in aspects, about 5 mL of the combination described herein can be used to cover about 5 cm². Thus can result in a matrix thickness of about 1 mm, which is the typical thickness of the matrix described herein.

Active agents may be incorporated into the matrices described herein. The active agents, when included, would be included in a therapeutically effectively amount to, for example, prevent infection, prevent infection, treat pain, and/or alleviate pain. Active agents include, but are not limited to, anti-inflammatoireuch as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, and antiinflammatory proteins or steroidal anti-inflammatory agents); antibiotics; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Other active agents include, without limitation: (1) immunosuppressants; glucocorticoids such as hydrocortisone, betamethisone, dexamethasone, flumethasone, isoflupredone, methylprednisolone, prednisone, prednisolone, and triamcinolone acetonide; (2) antiangiogenics such as fluorouracil, paclitaxel, doxorubicin, cisplatin, methotrexate, cyclophosphamide, etoposide, pegaptanib, lucentis, tryptophanyl-tRNA synthetase, retaane, CA4P, AdPEDF, VEGF-TRAP-EYE, AG-103958, Avastin, JSM6427, TG100801, ATG3, OT-551, endostatin, thalidomide, becacizumab, neovastat; (3) antiproliferatives such as sirolimus, paclitaxel, perillyl alcohol, farnesyl transferase inhibitors, FPTIII, L744, antiproliferative factor, Van 10/4, doxorubicin, 5-FU, Daunomycin, Mitomycin, dexamethasone, azathioprine, chlorambucil, cyclophosphamide, methotrexate, mofetil, vasoactive intestinal polypeptide, and PACAP; (4) antibodies; drugs acting on immunophilins, such as cyclosporine, zotarolimus, everolimus, tacrolimus and sirolimus (rapamycin), interferons, TNF binding proteins; (5) taxanes, such as paclitaxel and docetaxel; statins, such as atorvastatin, lovastatin, simvastatin, pravastatin, fluvastatin and rosuvastatin; and (6) antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B and silver salts such as chloride, bromide, iodide and periodate. It will be understood that the active agent may be present in the matrices described herein in any amount, typically from about 0.1% to about 30% by weight, such as from about 0.1%, about 1%, about 5%, about 10%, about 15%, about 20%, or about 25% to about 1%, about 5%, about 10%, about 15%, about 20%, about 25% or about 30% by weight.

Other chemical mediators, such as growth factors, interferons, interleukins, chemokines, monokines, hormones, and angiogenic factors, which can help with the wound healing process may also be incorporated into the matrices described herein and/or their production may be facilitated by the use of the matrix described herein when the matrix described herein is used for treating wounds. These factors include, but are not limited to, basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1a), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors—alpha and -beta (TGF-α. and TGF-β), interleukin-8 (IL-8), granulocyte-macrophage colony stimulating factor (GM-CSF), interleukins, and interferons. Commercial preparations of the foregoing are available from, for example, R & D Systems, Minneapolis, Minn.; Biovision, Inc, Mountain View, Calif.; ProSpec-Tany TechnoGene Ltd., Rehovot, Israel; and Cell Sciences™., Canton, Mass. In other aspects, the matrix described herein may also be used as a drug delivery carrier for biologics, for example, serum, plasma, platelet rich plasma, exosomes, serum proteins, etc.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way in the remainder of the disclosure.

### EXAMPLES

### Example 1: Preparation of a Degradable Hydrogel

(i) Preparation of a Hydrogel from a Non-Photopolymerizable Prepolymer

### Methods:

Hydrogels were rendered degradable through the covalent incorporation of the collagenase-sensitive peptide GGGPQGIWGQGK. Briefly, the peptide was dissolved in about 50 mM NaHCO₃(about pH 8.0) and reacted with about 3.4 kDa acryl-PEG-SVA in about a 1:2 (peptide: PEG-SVA) molar ratio at about 4° C. overnight to attach PEG on both ends of the peptide sequence (PEG-peptide-PEG). The resulting product, acrylate-PEG-peptide-PEG-acrylate was dialyzed, lyophilized, and reconstituted in sterile water to achieve about 10% (w/v) solution (S1). Hyaluronic acid and gelatin were each thiolated to produce thiol-modified hyaluronic acid and thiol-modified gelatin, respectively and a solution (S2) was prepared containing about 1 mg/mL thiol-modified hyaluronic acid, about 1 mg/mL thiol-modified gelatin and about 25 μg/mL tropoelastin. S1 and S2 were combined in about 1:1 ratio to yield the degradable hydrogel referred to as PEGScarX.

(ii) Preparation of Hydrogel from a Photopolymerizable Prepolymer

### Methods:

The prepolymer was prepared as in Example 1 and the resulting product, acrylate-PEG-peptide-PEG-acrylate was dialyzed, lyophilized, and reconstituted in sterile water to achieve about 10% (w/v) solution (S1) as described in Example 1. Triethanolamine, eosin-Y and n-vinylpyrrolidone were added to S1. S2 was also prepared as described in Example 1, except S2 did not contain tropoelastin. In this example, S2 contained only 1 mg/mL thiol-modified hyaluronic acid and 1 mg/mL thiol-modified gelatin. S1 was combined with S2 in about 1:1 ratio and exposed to white light for about 20 seconds to yield the hydrogel referred to as PEGScarX.

### Results:

Combination of S1 and S2 resulted in substantially instantaneous hydrogel in situ polymerization in about less than 1 minute when approximately equal ratios of 10% (w/v) S1 was combined with S2.

### Example 2 Assessment of Hydrogel Rheological Properties and Swelling/Degradation Kinetics

### Methods:

The PEGScarX hydrogel was prepared as described in Example 1. Integra® dermal regenerative matrix (DRM) was purchased and the PEGScarX and DRM were assayed in respect of rheological properties and degradation kinetics. Rheological properties of PEGScarX were studied using TA instruments HR-3 Discovery Hybrid Rheometer. Hydrogel degradation kinetics were determined by swelling the PEGScarX hydrogels for about 24 hours in phosphate buffered saline (PBS) at about 37° C. and then incubated with about 1 mg/ml collagenase at about 25° C. The change in wet weight was measured over time. All data are reported as mean±standard error of the mean (SEM). Statistical significance between treatments were determined by conducting a one-way analysis of variance (ANOVA) followed by Fisher's Least Significant Difference (LSD) post-hoc analysis. P<0.05 was considered significant.

### Results:

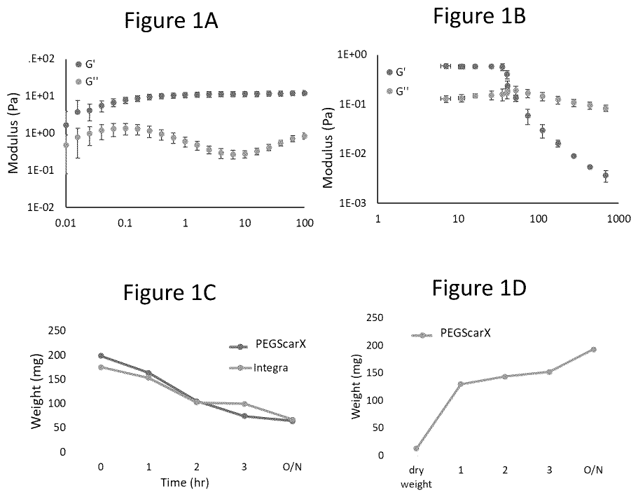

As shown in FIGS. 1A-1B, rheological analyses demonstrated that PEGScarX loses its solid behavior and yields after about 20% to about 30% strain material demonstrating compliant behavior. Moreover, as shown in FIG. 1C, similar degradation for PEGScarX and commercially available Integra® dermal regenerative matrix (DRM) was also noted. Finally, as shown in the FIG. 1D, the swelling kinetics of the PEGScarX were determined to be a 15.4±2.06 fold increase in weight compared to dry weight.

### Example 3 Assessment of Hydrogel Biocompatibility

Methods: The PEGScarX hydrogel was prepared as described in Example 1 and assayed for biocompatibility properties. For biocompatibility studies, burn-derived mesenchymal stromal cell (BDMSC) were used. The BDMSC were isolated and expanded using the following methods. Surgically debrided burn tissue was transferred to sterile containers. Upon receipt in the research laboratory, tissue was trimmed aseptically inside a biosafety cabinet to remove any excessive fat with the sterile scissors, scalpel and forcep. The trimmed tissue was washed in about 70% ethanol followed by two washes in phosphate buffered saline (PBS) containing about 1% Ab/Am. The tissue was immediately submerged in High Glucose Dulbecco's Modified Eagle Medium (DMEM)/F-12 without fetal bovine serum (FBS). About 3 g to about 5 g of trimmed tissue was divided into multiple falcon tubes. For BDMSC extraction, the tissue was minced into fine pieces (<1 mm²) using surgical scalpels and scissors. BDMSCs were isolated through a multi-step enzymatic process. The chopped pieces were first treated with CTS™ TrypLE™ Select Enzyme on a rotary shaker at about 37° C. for about 15 minutes, followed by straining with about a 40 μm cell strainer. The retained tissue was digested further with a collagenase digestion mixture containing about 702 U/mL of collagenase I (ThermoFisher Scientific) and about 0.81 U/mL dispase II (Roche) with stirring at about 600 rpm at about 37° C. for about 90 min, and final cell dissociation with DNase I (Roche) on a rotary shaker at about 37° C. for about 10 min followed by cell straining with about 100 um cell strainer. The final flow through collected was spun down at about 1400 rpm, containing the isolated BDMSCs. Initially isolated BDMSCs were cultured in T75 flasks containing CTS™ KnockOut™ DMEM/F-12 media (Gibco) supplemented with about 10% FBS and about 2% of Antibiotic/Antimycotic inside a humidified incubator at about 37° C. with about 5% CO₂. Once a confluency of about 80% to about 90% was achieved, the cells was trypsinized using CTS™ TrypLE™ Select Enzyme and further expanded in T175 flasks. Once the BDMSCs were isolated and expanded as described, about 1× about 10⁶ burn-derived mesenchymal stem cells (BDMSCs) were embedded in PEGScarX to assess cell viability, adhesion and spreading. All data are reported as mean±standard error of the mean (SEM). Statistical significance between treatments were determined by conducting a one-way analysis of variance (ANOVA) followed by Fisher's Least Significant Difference (LSD) post-hoc analysis. P<0.05 was considered significant.

### Results:

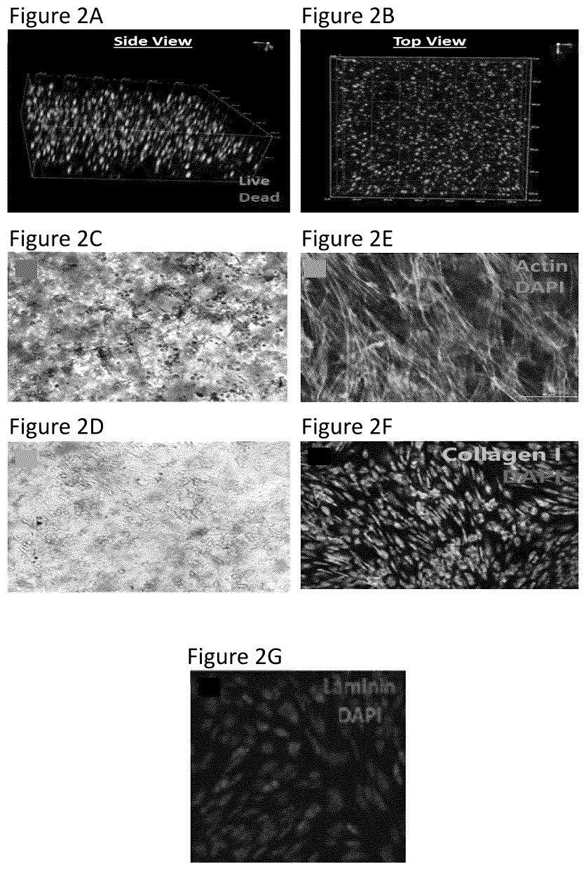

As shown in FIG. 2A, BDMSCs embedded in PEGScarX demonstrated no hypoxic centers or cell death in the geometrical center. The hydrogel was also shown to be conducive to cell adhesion/spreading on day 5 and day 21 post-encapsulation (FIG. 2B-2C) as shown by positive immunofluorescent staining for actin, collagen and laminin (FIG. 2D-2F).

### Example 4 Assessment of Hydrogel Contraction

### Methods:

The PEGScarX hydrogel was prepared as described in Example 1 and the contractile properties of the PEGScarX were assessed. Contraction assays were conducted with or without fibroblasts in vitro to measure any reduction in area over time. Images were taken with a ruler placed next to the hydrogel. The images were then traced on NIH FIJI software to measure the area. All data are reported as mean±standard error of the mean (SEM). Statistical significance between treatments were determined by conducting a one-way analysis of variance (ANOVA) followed by Fisher's Least Significant Difference (LSD) post-hoc analysis. P<0.05 was considered significant.

### Results:

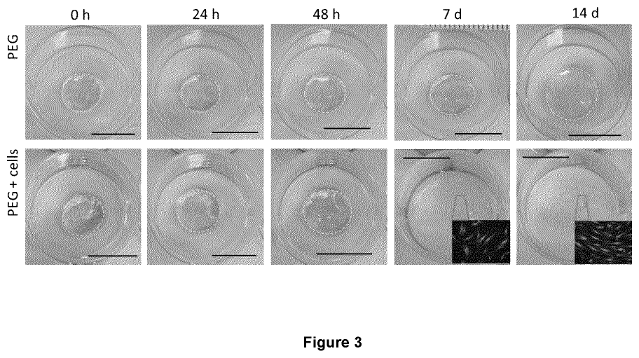

Collagen-containing matrices demonstrate contraction over time. PEGScarX did not demonstrate any contraction with or without cells in vitro. PEGScarX degraded over time due to cell-secreted matrix metalloproteinases (MMPs). As shown in FIG. 3, PEGScarX began to degrade by cell-secreted MMPs by day 7 without loss of cell viability (inset) and the released cells attached to the underlying tissue culture plates.

### Example 5 Assessment of Factor Secretion from PEGScarX Embedded BDMSCs

### Methods:

PEGScarX was prepared as described in Example 1. BDMSCs were isolated, expanded and embedded in PEGScarX as described in Example 3. The cell culture supernatant was collected at 48 hours and cytokines, chemokines, growth factors, and immunomodulatory proteins were detected using the HCYTOMAG-60 K MILLIPLEX MAP Human Cytokine/Chemokine Magnetic Bead Panel-Immunology Multiplex Assay (EMD Millipore Corporation, Germany).

### Results:

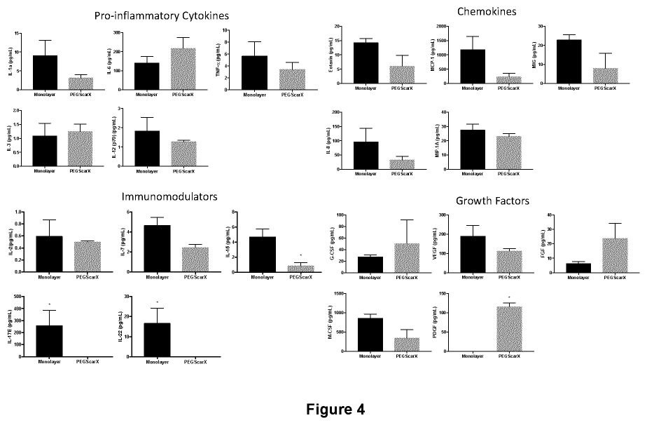

As shown in FIG. 4, no statistically significant differences were observed in secretion of pro-inflammatory cytokines IL-1α, IL6, TNF-α, IL-3, IL-12 or chemokines, eotaxin, MCP-1, MIG, IL-8 or MIP-1α, between cell cultured in monolayers or embedded in PEGScarX. Among immunomodulators, IL-15, IL-17E (IL-25) and IL-22 were secreted at significantly lower level from cells embedded in PEGScarX compared to monolayer cultured cells. Finally, no differences were observed in growth factor secretion with the exception of PDGF which was only secreted from cells embedded in PEGScarX.

### Example 6 In Vivo Testing: Thermal Injury in a Pig Animal Model

### (i) Wound Preparation and Processing

### Methods:

All animal procedures were performed on female Yorkshire pigs (strain; x-y weight, Farm, location, province) in accordance with protocols approved by the Animal Policy and Welfare Committee of the University of Toronto, Sunnybrook Research Institute and Sunnybrook Health Sciences Centre Animal Care and Use Committee. All pigs were acclimatized for at least 1 week prior to thermal injury. Under anesthesia (ketamine at about 0.2 mg/kg sc. combined with Atropine about 0.5 to about 1.0 mg depending on the heart rate and isoflurane about 5%/I/O₂ intubation) and analgesia (Buprenorphine about 0.05 mg/kg subcutaneous (sc.), 5×5 cm dorsal full-thickness burns were created using a heated aluminum device (about 200° C.) for 20 seconds under a constant force of about 4N measured using a digital force gauge (Mark-10 Corporation). About a 5 cm interspace was maintained between adjacent wounds. The wounds were covered with paraffin gauze and wet to dry gauze dressings that were kept in place by an adhesive bandage, and an elastic stocking porcine suit. Burned skin was removed after about 48 hours by inflicting full thickness excisional wounds (post-excision day 0).

### (ii) Analysis of Wound Healing

### Methods:

PEGScarX were prepared as described in Example 1 and S1 and S2 were combined for in situ polymerization onto burn wounds of the thermally injured pigs produced above. In addition, Integra® dermal regenerative matrix (DRM) was purchased and used for comparative analysis with the PEGScarX in these in vivo studies. Wounds were photographed at each dressing changed for gross appearance and wound area. Wound area was measured using NIH ImageJ software. Wound closure was calculated as percent area of original wound. The wound closure rate was calculated using the following equation: % Wound closure=[(Day 0 wound area−Day X wound area)/Day 0 wound area]×100.

Gross wound examination was conducted by taking photographs of the wounds at post-injury days 0, 4, 7, 10, 14, 18, 21, 26, 32 and 40 with and without the PEGScarX application. The photographs were used to score granulation tissue formation and the Vancouver scar scale by scoring for vascularity, pigmentation, pliability, and height. Evaluation for scarring was done by external blinded burn surgeons.

(iia) Histochemical and Immunohistochemical Analysis:

### Methods:

Wound tissue, with or without treatment with PEGScarX or DRM, was explanted at sacrifice and trimmed, followed by fixation in about 10% formalin for about 48 h at room temperature, then stored in about 70% ethanol at about 4° C. until serial sectioning (about 5 μm thick) at SRI Histology Core. Histological sections were stained with hematoxylin and eosin (H & E) for morphological analysis and Masson's Trichrome for collagen density. Immunohistochemical analysis was conducted to analyze wound contraction (alpha smooth muscle actin; α-SMA), neovascularization (CD 31) and wound inflammation (CD 11b). Stained slides were imaged under Leica light microscope (LEICADM 2000 LED) and analyzed with the evaluator blinded to treatment/control conditions. Histological images were analyzed on ImageJ software by thresholding RGB images and converted to gray scale to determine collagen density, % area positive for a-SMA, no. of blood vessels and no. of inflammatory cells.

All data for this Example were reported as mean±standard error of the mean (SEM). Statistical significance between treatments were determined by conducting a one-way analysis of variance (ANOVA) followed by Fisher's Least Significant Difference (LSD) post-hoc analysis. P<0.05 was considered significant.

### Results:

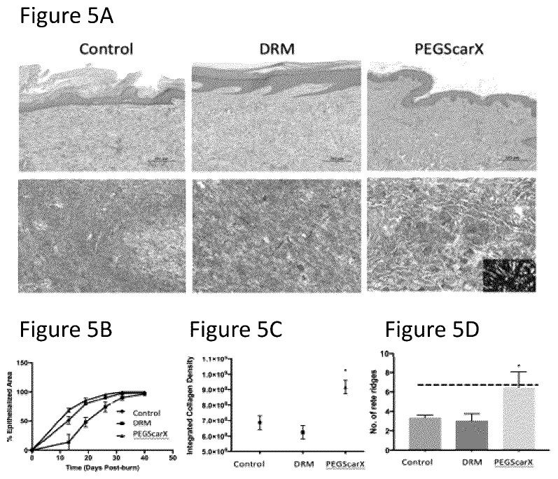

As shown in FIGS. 5A-5D, PEGScarX was found to promote faster healing kinetics and improved wound histological features compared to commercial dermal regenerative matrix (DRM). As shown in FIG. 5A, the H & E and Masson's trichrome histological sections demonstrated improved healing in wounds treated with PEGScarX as compared to DRM treated wounds or untreated controls. Collagen fibers of the PEGScarX treated wounds were more basket-weave like (inset) similar to uninjured skin. As shown in FIG. 5B, PEGScarX treated wounds had faster re-epithelialization kinetics as compared to DRM treated wounds or untreated controls. Finally, as shown in FIGS. 5C and 5D, collagen density (FIG. 5C) and the number of rete ridges (FIG. 5D) were both significantly higher in PEGScarX treated wounds as compared to DRM treated wounds and untreated controls. Of note the number of rete ridges was comparable to that of uninjured skin (dotted line; FIG. 5D).

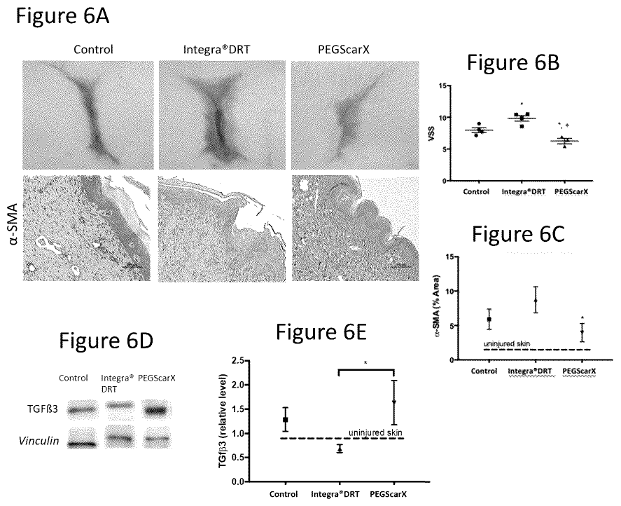

As shown in FIG. 6A, gross appearance of wound scar demonstrates significantly reduced scarring (top) and lower expression of α-SMA (bottom) in PEGScarX wounds. As shown in FIGS. 6A (bottom) and 6C, respectively, α-SMA immunohistochemical staining with significantly lower contraction in PEGScarX wounds (3.96±2.66), while α-SMA intensities were 5.90±4.65 and 8.74±5.99 for untreated and DRM controls, respectively. Quantification of α-SMA positive area demonstrates significantly lower contracting myofibroblasts in PEGScarX, In addition, Vancouver Scar Scores are significantly lower in PEGScarX treated wounds compared to no treatment controls and commercial Integra DRT treated wounds (FIG. 6B). The scar scores in PEGScarX treated wounds (6.26±0.76) compared to no treatment (7.98±0.78) and DRM treatment (9.84±0.89) controls. Finally, as shown in FIGS. 6D and 6E, anti-scarring mediator TGFβ3 levels were significantly higher in PEGScarX treated wounds (1.63±0.79) compared to 1.28±0.78 and 0.69±0.23 in untreated and DRM treated wounds, respectively.

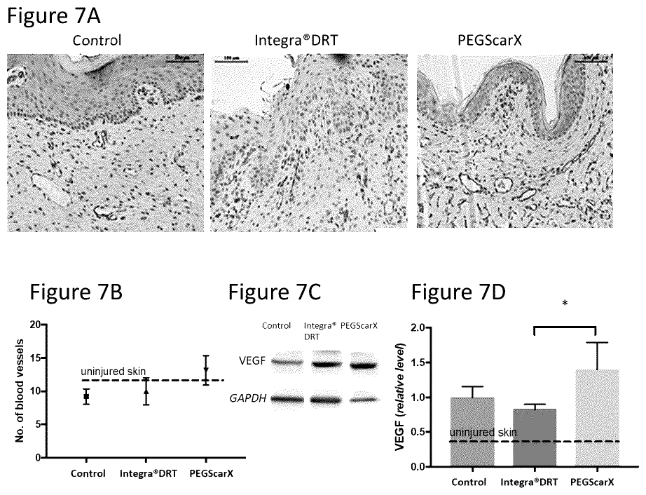

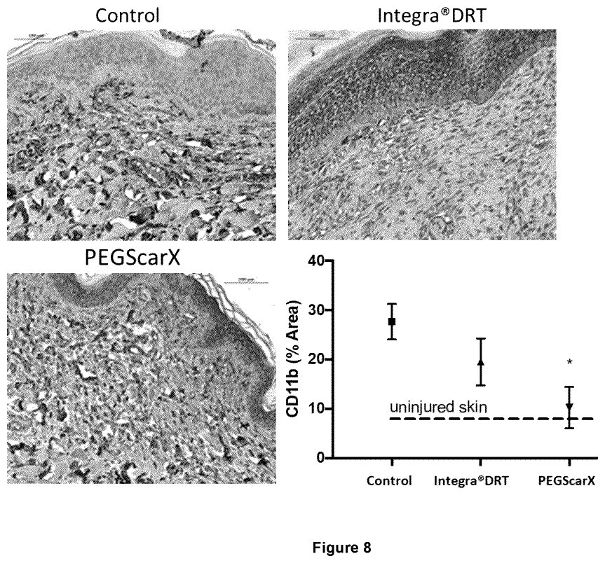

Finally, as shown in FIGS. 7A and 7C, Western blot analysis of VEGF in wound sections demonstrated significantly increased VEGF levels in PEGScarX treated wounds, with values corresponding to 1.39±0.79, 0.99±0.49, and 0.82±0.24 in PEGScarX, untreated wounds and DRM treated wounds, respectively. In addition, it was found that PEGScarX promotes neovascularization since IHC anti-CD31 stained sections demonstrate significantly higher number of blood vessels found in the PEGScarX treated wounds as compared to DRM treated wounds and untreated controls. Moreover, as shown in FIGS. 8A-8D, wound inflammation was lowest in the PEGScarX treated wounds as compared to controls and DRM treated wounds (FIGS. 8A-8C), and the lowest expression of the immune cell marker CD11b, a marker of inflammation, was seen in PEGScarX treated wounds as compared to DRM treated wounds or untreated controls (FIG. 8D). Thus, there were fewer CD11b+ cells, indicating reduced inflammation, in the PEGScarX treated wound.

### Overall Discussion:

The Examples provided herein demonstrate an instantaneous, in-situ polymerizable modular matrix generated from thiol-Michael addition click reaction for biotherapeutic delivery to burn wounds without the need of a light source in a non-specialized setting. The degradable PEG hydrogel construct exemplified herein by integrating protease cleavable peptide sequences in the polymer backbone with acrylate side chains can allow instantaneous hydrogel formation when reacted with thiol-modified biomolecules. These results suggest the formation of a modular hydrogel system which can allow for the addition of biomolecules that support skin regeneration.

Scar outcomes are a result of an interplay of wound angiogenesis, fibroblast-to-myofibroblast transition, expression of transforming growth factor-beta (TGF-β), deposition and subsequent remodelling of ECM, and alignment of collagen fibres. The Examples provided herein demonstrate lower expression of a-smooth muscle actin in PEGScarX treated wounds compared to non-treated controls. This may be indicative of a reduction of fibrosis and attenuation of contraction. The Examples also demonstrated high VEGF and TGF-β3 levels in PEGScarX treated wounds which is suggests that PEGScarX can minimize scar formation, enhance angiogenesis and/or promote skin regeneration.

Thus, using materials engineering and approaches from regenerative medicine the exemplified hydrogel can allow for growth inductive to cells from the surrounding uninjured skin, can be cellularly degradable with tailored substrate stiffness, can provide a scaffold for endogenous cellular infiltration, can promote granulation tissue formation, can be conducive to cell proliferation, cell matrix deposition and can include biofactors that support anti-scarring. In addition to the aforementioned properties, the exemplified hydrogel may allow for critical, early-stage trauma care at the point of injury or a pre-hospital setting by non-specialized service members.

### Example 7 Assessment of Hydrogel as a Donor Site Dressing

### (i) Production and Assessment of Donor Site Dressings

### Methods:

Donor site dressings PEG-tHG and dPEG-tHG were prepared. Briefly, dPEG-tHG were prepared as follows: The collagenase-sensitive peptide GGGPQGIWGQGK was dissolved in about 50 mM NaHCO₃ (about pH 8.0) and reacted with about 3.4 kDa acryl-PEG-SVA in about a 1:2 (peptide: PEG-SVA) molar ratio at about 4° C. overnight to attach PEG on both ends of the peptide sequence (PEG-peptide-PEG). The resulting product, acrylate-PEG-peptide-PEG-acrylate was dialyzed, lyophilized, and reconstituted in sterile water to achieve about 10% (w/v) solution (S1). To this, about 1.5% v/v triethanolamine/HEPES-buffered saline (about pH 7.4), about 37 mM 1-vinyl-2-pyrrolidinone, about 0.1 mM eosin-Y were added. Thiol-modified hyaluronic acid and thiol-modified gelatin, was prepared containing about 1 mg/mL thiol-modified hyaluronic acid, about 1 mg/mL thiol-modified gelatin and about 25 μg/mL tropoelastin to prepare solution S2. S1 and S2 were combined in about 1:1 ratio, pipetted into custom-made molds of about 1 cm to about 2 cm thickness gap and exposed to white light for about 20 seconds to yield dPEG-tHG. PEG-tHG was prepared in a similar fashion except PEGDA at about 10 kDA MW was used in S1 instead of acrylate-PEG-peptide-PEG-acrylate. Allevyn and Xeroform were purchased for comparison to the prepared donor site dressings. Gross appearance of each of the donor site dressings was evaluated before and after swelling. In addition, swelling kinetics and ratios were also determined. Donor site dressing degradation kinetics were determined by swelling the donor site dressing for about 24 hours in PBS at about 37° C. The change in wet weight was measured over time. PEG-tHG and dPEG-tHG were dried in a lyophilizer for about 48 hours to get the dry weight.

### Results:

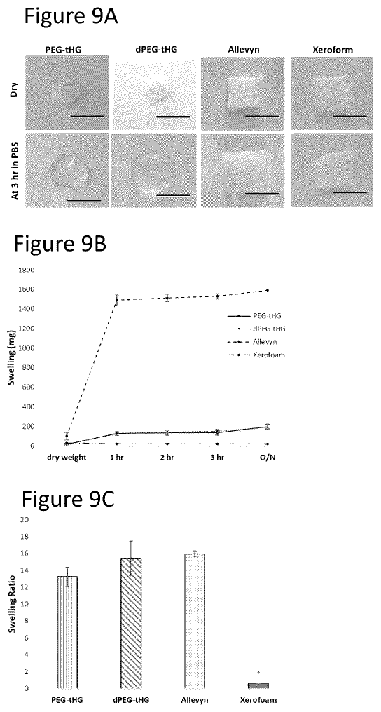

As shown in FIG. 9, Allevyn demonstrated the highest swelling or increase in weight within the first hour corresponding to a swelling ratio of about 15.95 while xeroform demonstrated minimal increase in wet volume with a swelling ratio of only about 0.6. dPEG-tHG and PEG-tHG demonstrated swelling ratios of about 15.4 and about 13.2, respectively.

### (ii) Assessment of Donor Site Dressing on Donor Site Wounds

### Methods:

The donor site dressings were prepared as described in Example 7. Assessments were conducted in a porcine model. Briefly, split thickness skin was excised using a hand-held humby knife to replicate harvest of donor skin as would be done for an autograft harvest in burn patients. Donor site dressings were applied to the wound areas and imaged at several time points to assess healing. (i). Gross appearance of donor site wounds was assessed. Images of wounds were taken with a ruler placed next to the wound. Changes in open wound area were measured on image J software to compute % wound closures. Donor site wound healing kinetics and Vancouver scar scales for donor site wound scary quality were determined. Wound scar assessment was conducted by utilizing the Vancouver Scar Scale by scoring for vascularity, pigmentation, pliability, and height. Evaluation for scarring was done by external blinded burn surgeons. In addition, histological evaluation of the donor site wounds was conducted, rete ridges and collagen density was quantified. Wound tissue was explanted at sacrifice and trimmed, followed by fixation in about 10% formalin for about 48 hours at about room temperature, then stored in about 70% ethanol at about 4 C until serial sectioning (about 5 μm thick) at SRI Histology Core. Histological sections were stained with hematoxylin and eosin (H & E) for morphological analysis and Masson's Trichrome for collagen density. Stained slides were imaged under Leica light microscope (LEICADM 2000 LED) and analyzed with the evaluator blinded to treatment/control conditions. Histological images were analyzed on ImageJ software by thresholding RGB images and converted to gray scale to determine collagen density.

### Results:

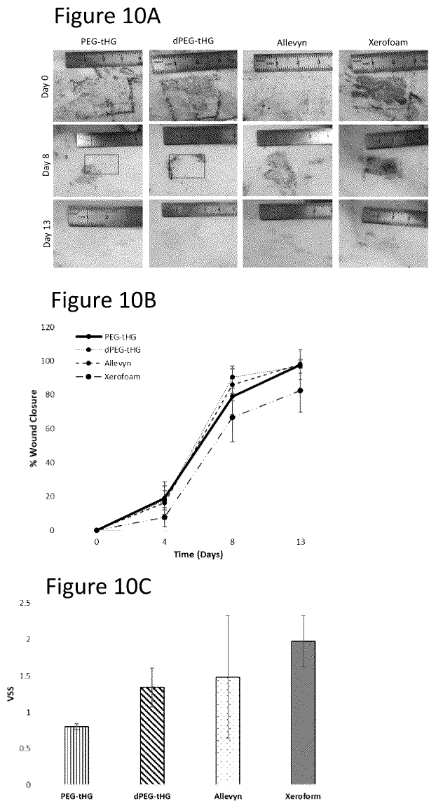

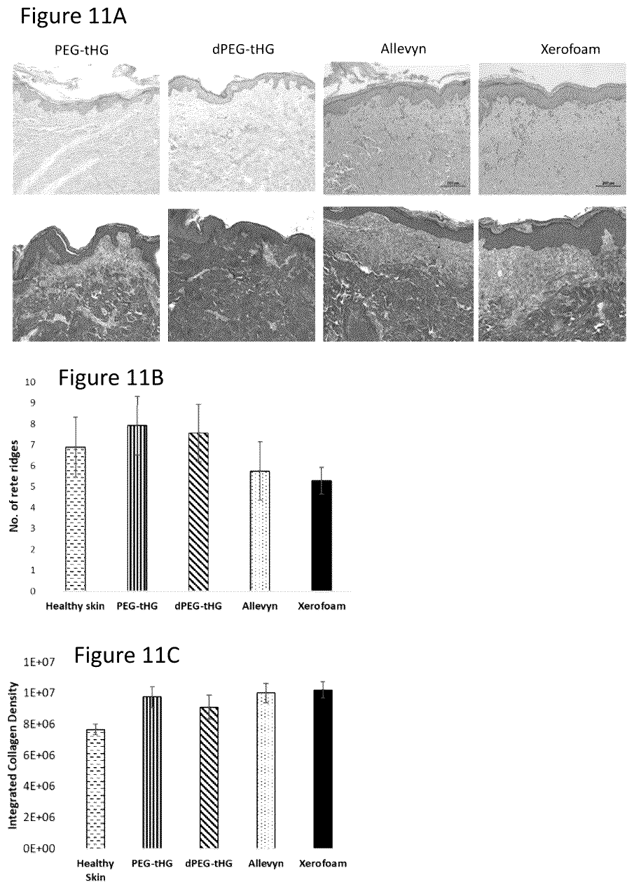

As shown in FIG. 10, the % wound closures were measured and results demonstrated fastest healing in split thickness skin wounds treated with dPEG-tHG. Xeroform demonstrated slowest wound healing. Vancouver Scar scores were lowest in PEG-tHG and dPEG-tHG demonstrating scar free healing. Split thickness wounds treated with PEG-tHG and dPEG-tHG demonstrated wound anatomical features similar to healthy skin with the presence of rete ridges (FIGS. 11A-11C). These wounds also demonstrated reduced presence of infiltrating cells compared to Allevyn and Xeroform treated wounds. Collagen stained sections further confirmed this finding by demonstrating presence of collagen bundles similar to healthy skin while Allevyn and Xeroform treated wounds were still not completely healed.

The above disclosure generally describes the present invention. Although specific terms have been employed herein, such terms are intended in a descriptive sense and not for purposes of limitation. All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A polyethylene glycol (PEG) prepolymer comprising a reaction product of an amine-reactive PEG and a protease degradable peptide, wherein the reaction product comprises the formula [Acryl-PEG-peptide-PEG-Acryl]ₙ, wherein n is greater than 0.

2-5. (canceled)

6. A combination comprising the PEG prepolymer of claim 1 and a thiolated biomolecule.

7-17. (canceled)

18. A polymeric wound matrix comprising:
a polyethylene glycol (PEG) prepolymer comprising a reaction product of an amine-reactive PEG and a protease degradable peptide, wherein the reaction product comprises the formula [Acryl-PEG-peptide-PEG-Acryl]ₙ, wherein n is greater than 0; and
a thiolated biomolecule, wherein the thiolated biomolecule is crosslinked to acrylate side chains of the PEG prepolymer to form the wound matrix.

19. The matrix of claim 18, wherein the amine reactive PEG is selected from acrylate-poly (ethylene glycol)-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succinimidyl succinate (PEG-SS) or acrylate-PEG-maleimide (PEG-MAL).

20. The matrix of claim 19, wherein the amine-reactive PEG is acrylate-PEG-SVA.

21. The matrix of claim 18, wherein:
(i) the peptide comprises or consists of the sequence GGGPQGIWGQGK (SEQ ID NO: 1), GGGIQQWGPGGK (SEQ ID NO: 2), or GGGGGIPQQWGK (SEQ ID NO: 3); or
(ii) the peptide comprises or consists of the sequence GGGPQGIWGQGK (SEQ ID NO: 1), or a fragment or variant thereof.

22-23. (canceled)

24. The matrix of claim 18, wherein the thiolated biomolecule comprises thiolated hyaluronic acid, thiolated gelatin, tropoelastin, or combinations thereof.

25. The matrix of claim 18, wherein the PEG prepolymer and the thiolated biomolecule are combined prior to use, for in situ, polymerization on a surface, optionally wherein the surface is a wound bed.

26. The matrix of claim 25, wherein:
(i) polymerization occurs in about 1 second to about 30 minutes;
(ii) polymerization occurs in about 10 seconds to about 20 seconds; or
(iii) polymerization does not require a light source and/or photoinitiator.

27-28. (canceled)

29. The matrix of claim 25, further comprising a photoinitiator, wherein the photoinitiator is added to the PEG prepolymer prior to combination with the thiolated biomolecule.

30. The matrix of claim 29, wherein the photoinitiator is selected from triethanolamine, ruthenium, eosin-Y and n-vinylpyrrolidone and their derivatives and mixtures thereof.

31-32. (canceled)

33. The matrix of claim 18, wherein;
(i) the matrix does not contract over time;
(ii) the matrix is biodegradable; or
(iii) the matrix supports cell viability.

34-35. (canceled)

36. The matrix of claim 18, further comprising cells, such as epidermal cells, including keratinocytes and fibroblasts, skin immune cells, endothelial cells, stem cells, such as induced pluripotent stem cells and mesenchymal stem cells, such as burn-derived mesenchymal stem cells, and/or products derived from said cells such as a secretome or exosomes from mesenchymal stem cells.

37. The matrix of claim 18, wherein:
(i) the matrix promotes wound regeneration and/or reduces scarring;
(ii) the matrix promotes increased anti-scarring mediator levels when the matrix is placed on a wound, optionally wherein the anti-scarring mediator is TGF-β3;
(iii) the matrix promotes granulation tissue formation and/or re-epithelization of a wound;
(iv) the matrix promotes neovascularization of a wound;
(v) the matrix reduces wound inflammation; or
(vi) the matrix treats and/or seals a wound.

38-44. (canceled)

45. The matrix of claim 18, wherein the matrix is comprised in a biodegradable wound dressing or a biodegradable or non-biodegradable donor site dressing.

46. The matrix of claim 18, wherein the matrix is a hydrogel.

47. A method of treating and/or sealing a wound of a subject, comprising applying the matrix of claim 18 to the wound, thereby treating and/or sealing the wound of the subject.

48. The method of claim 47, wherein the wound comprises burns, partial and full-thickness wounds, acute wounds, chronic wounds, pressure ulcers, venous ulcers, arterial ulcers, diabetic ulcers, chronic vascular ulcers, draining wounds, tunneled or undermined wounds, surgical wounds, donor site wounds, or trauma wounds.

49-50. (canceled)

51. A bio-ink formulation comprising:
a polyethylene glycol (PEG) prepolymer comprising a reaction product of an amine-reactive PEG and a protease degradable peptide, wherein the reaction product comprises the formula [Acryl-PEG-peptide-PEG-Acryl]n, wherein n is greater than 0, and
a thiolated biomolecule, wherein the thiolated biomolecule crosslinks acrylate side chains of the PEG prepolymer; and
wherein the bio-ink formulation is used for skin regeneration.

52. A method of making a polymeric matrix, comprising combining the PEG prepolymer of claim 1 with a thiolated biomolecule and crosslinking acrylate side chains of the PEG prepolymer with the thiolated biomolecule, thereby forming the polymeric matrix.

53. The bio-ink formulation of claim 51, wherein:
(i) the amine reactive PEG is selected from acrylate-poly (ethylene glycol)-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succinimidyl succinate (PEG-SS) or acrylate-PEG-maleimide (PEG-MAL);
(ii) the peptide comprises or consists of the sequence GGGPQGIWGQGK (SEQ ID NO: 1), GGGIQQWGPGGK (SEQ ID NO: 2), or GGGGGIPQQWGK (SEQ ID NO: 3); and/or
(iii) the thiolated biomolecule has wound regeneration and/or anti-scarring properties and/or wherein the thiolated biomolecule comprises thiolated hyaluronic acid, thiolated gelatin, tropoelastin, or combinations thereof.
